(19) United States
(12) Patent Application Publication
(10) Pub. No.: US 2024/0180454 A1
(43) Pub. Date: Jun. 6, 2024

(54) DETECTION DEVICE

(71) Applicant: Japan Display Inc., Tokyo (JP)

(72) Inventors: Kento HIMOTO, Tokyo (JP); Takashi NAKAMURA, Tokyo (JP); Gen KOIDE, Tokyo (JP)

(21) Appl. No.: 18/442,219

(22) Filed: Feb. 15, 2024

Related U.S. Application Data
(63) Continuation of application No. PCT/JP2022/032279, filed on Aug. 26, 2022.

(30) Foreign Application Priority Data
Sep. 1, 2021 (JP) 2021-142675

(51) Int. Cl. A61B 5/1455

(52) U.S. Cl. CPC A61B 5/14551; A61B 2562/185

(57) ABSTRACT

According to an aspect, a detection device includes: a base member having a first principal surface; a detection area provided to the first principal surface; a sensor including a plurality of photodiodes; gate lines; signal lines; and a light emission device. The light emission device includes light sources. A lit area in which the light emission device is lit up is a portion of the detection area overlapping the light sources. A portion of the detection area not overlapping the lit area is an unlit area in which the light emission device is not lit up. Photodiodes that overlap the unlit area are selected from among the photodiodes, and amounts of light received by the selected photodiodes are read.

### CROSS-REFERENCE TO RELATED APPLICATION

This application claims the benefit of priority from Japanese Patent Application No. 2021-142675 filed on Sep. 1, 2021 and International Patent Application No. PCT/JP2022/032279 filed on Aug. 26, 2022, the entire contents of which are incorporated herein by reference.

### BACKGROUND

### 1. Technical Field

What is disclosed herein relates to a detection device.

### 2. Description of the Related Art

Detection devices that include a light source and a sensor have been developed in recent years to detect a vascular pattern of, for example, veins located in a finger or a thumb, a wrist, or a foot. In a detection device of Japanese Translation of PCT International Application Publication Laid-open No. 2020-529695, the light source and the sensor are arranged so as to interpose an object to be detected therebetween. In such a detection device, light is emitted from the light source to the skin and enters the body. The light then passes through the blood, muscular tissues, and the like inside the body, and is further transmitted outside the body to be received by the sensor.

When the light source and the sensor are placed in the same direction with respect to the object to be detected, the sensor receives light reflected from the skin or shallow portions in the body. This light reflected from the skin or the shallow portions in the body has not passed through the blood. Therefore, this light is noise in detecting the vascular pattern, and the vascular pattern may not be accurately detected.

For the foregoing reasons, there is a need for a detection device capable of accurately detecting a vascular pattern with a light source and an optical sensor placed in the same direction with respect to an object to be detected.

### SUMMARY

According to an aspect, a detection device includes: a base member having a first principal surface; a detection area provided to the first principal surface; a sensor including a plurality of photodiodes that are arranged in the detection area and arranged in a first direction parallel to the first principal surface and a second direction parallel to the first principal surface and intersecting the first direction; a plurality of gate lines that extend in the first direction and are coupled to the photodiodes; a plurality of signal lines that extend in the second direction and are coupled to the photodiodes; and a light emission device configured to emit light through gaps between the photodiodes in a direction in which the first principal surface faces. The light emission device includes a plurality of light sources. A lit area in which the light emission device is lit up is a portion of the detection area overlapping the light sources when viewed in a third direction intersecting the first direction and the second direction. A portion of the detection area not overlapping the lit area is an unlit area in which the light emission device is not lit up. Photodiodes that overlap the unlit area when viewed in the third direction are selected from among the photodiodes, and amounts of light received by the selected photodiodes are read.

### BRIEF DESCRIPTION OF THE DRAWINGS

FIG. 1 is a sectional view illustrating a schematic sectional configuration of a detection device according to a first embodiment of the present disclosure;

FIG. 2 is a plan view illustrating the detection device according to the first embodiment;

FIG. 3 is a block diagram illustrating a configuration example of the detection device according to the first embodiment;

FIG. 4 is a circuit diagram illustrating the detection device;

FIG. 5 is a circuit diagram illustrating a plurality of partial detection areas;

FIG. 6 is a timing waveform diagram illustrating an operation example of the detection device;

FIG. 7 is a timing waveform diagram illustrating an operation example during a read period in FIG. 6;

FIG. 8 is a magnified schematic configuration diagram of a sensor;

FIG. 9 is a sectional view along IX-IX′ of FIG. 8;

FIG. 10 is a plan view illustrating a configuration example of a light emission device according to the first embodiment;

FIG. 11 is a plan view illustrating a reading area at a first lighting time in the first embodiment;

FIG. 12 is a plan view illustrating the reading area at a second lighting time in the first embodiment;

FIG. 13 is a sectional view at the first lighting time in the detection device of the first embodiment;

FIG. 14 is a plan view obtained by viewing a detection device according to a second embodiment of the present disclosure in plan view;

FIG. 15 is a plan view obtained by viewing a detection device according to a third embodiment of the present disclosure in plan view;

FIG. 16 is a plan view obtained by viewing a detection device according to a fourth embodiment of the present disclosure in plan view;

FIG. 17 is a flowchart for detecting a blood oxygen saturation level in a detection device according to a fifth embodiment of the present disclosure;

FIG. 18 is a plan view obtained by viewing the detection device at a first step in plan view in the fifth embodiment;

FIG. 19 is a plan view obtained by viewing the detection device at a third step in plan view in the fifth embodiment; and

FIG. 20 is a sectional view of a detection device according to a modification.

### DETAILED DESCRIPTION

The following describes modes (embodiments) for carrying out the present disclosure in detail with reference to the drawings. The present disclosure is not limited to the description of the embodiments to be given below. Components to be described below include those easily conceivable by those skilled in the art or those substantially identical thereto. In addition, the components to be described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### First Embodiment

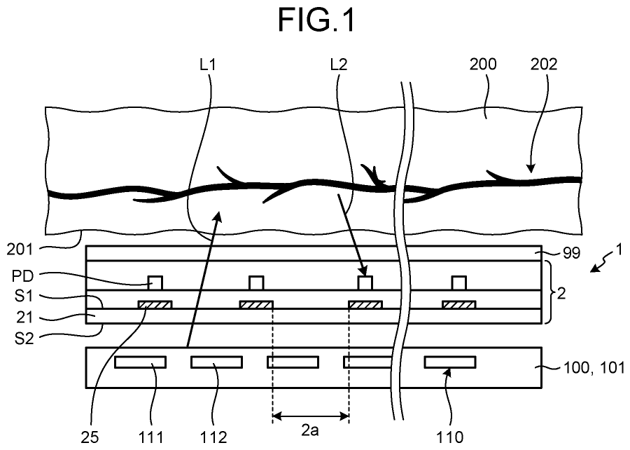

FIG. 1 is a sectional view schematically illustrating a configuration of a detection device according to a first embodiment of the present disclosure. First, a basic configuration of a detection device 1 will be described. As illustrated in FIG. 1, the detection device 1 includes an array substrate 2, a cover member 99, and a light emission device 100 (backlight 101). The backlight 101, the array substrate 2, and the cover member 99 are stacked in this order in a direction orthogonal to a first principal surface S1 of the array substrate 2.

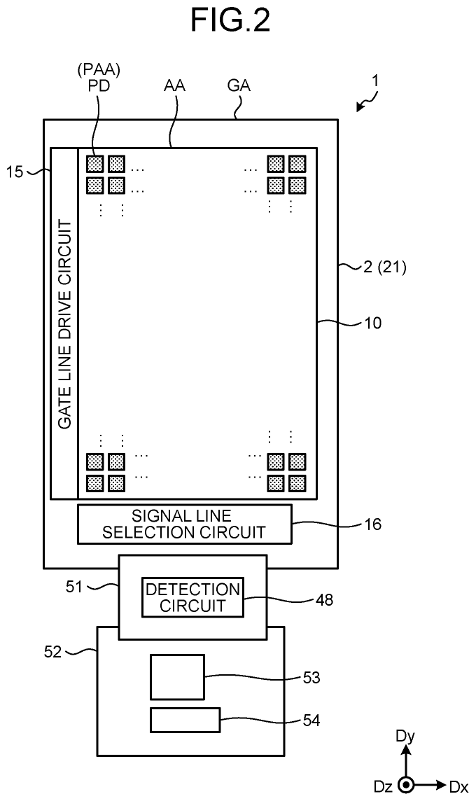

The array substrate 2 includes a base member 21 as a base. The base member 21 has the first principal surface S1 and a second principal surface S2 that is a surface opposite to the first principal surface S1. The first principal surface S1 has a detection area AA and a peripheral area GA (refer to FIG. 2). The detection area AA is provided with a plurality of light shields (light shield layers) 25 and a plurality of photodiodes PD. Thus, the detection area AA is divided into light-blocking areas and light-transmitting areas 2a. The light-blocking areas are areas that overlap the light shields 25 and various types of wiring, and the light-transmitting areas 2a are areas that do not overlap any of the light shields 25 and the various types of wiring.

The cover member 99 is a member for protecting the array substrate 2 and covers the array substrate 2. During detection, the cover member 99 faces an object to be detected 200. The cover member 99 is a glass substrate, for example. In the present disclosure, the cover member 99 is not limited to a glass substrate, but may be a resin substrate, for example, and may have a configuration composed of a plurality of layers made by stacking these substrates. The cover member 99 of the present embodiment is bonded to the array substrate 2 by an adhesive layer, which is not illustrated. The adhesive layer may be absent in the present disclosure. In the present disclosure, the array substrate 2 need not have the cover member 99. In that case, a protective layer such as an insulating film is provided on the surface of the array substrate 2.

The light emission device 100 is a device that emits light through gaps between the photodiodes PD in a direction in which the first principal surface S1 faces. The light emission device 100 of the present disclosure is the backlight 101. The backlight 101 is disposed so as to face the second principal surface S2 of the base member 21. The backlight 101 includes a plurality of light sources 110.

The backlight 101 emits light toward the second principal surface S2 of the base member 21. Light L1 emitted from the backlight 101 passes through the light-transmitting area 2a of the array substrate 2 and is emitted to the object to be detected 200. The light L1 enters the object to be detected 200 through skin 201 of the object to be detected 200 and passes through blood 202 and muscle tissues. The light L1 is reflected in the object to be detected 200. Reflected light L2 that has been reflected therein is emitted outside the object to be detected 200. The photodiodes PD of the detection device 1 receive the reflected light L2. Thus, information on the blood 202 is acquired.

The light sources 110 include first light sources 111 and second light sources 112. The first light sources 111 of the present embodiment emit infrared light having a wavelength of 880 nm. The second light sources 112 emit red light having a wavelength of 665 nm. During the detection, the first and the second light sources 111 and 112 are alternately lit up. Therefore, the photodiodes PD alternately receive the reflected light L2 of the infrared light and the red light.

The reflected light L2 of the infrared light contains information for detecting a vascular pattern. Red blood cells contained in the blood contain hemoglobin. The infrared light emitted from the first light sources 111 can be easily absorbed by hemoglobin. In other words, the coefficient of absorption of infrared light by hemoglobin is higher than that by the other portions in the body. Therefore, the vascular pattern of, for example, veins can be detected by reading the amounts of light received by the photodiodes PD and identifying locations where the amount of the received reflected light L2 of the infrared light is relatively small.

The reflected light L2 of the infrared light and the red light contains information for measuring an oxygen saturation level in the blood (hereinafter, referred to as blood oxygen saturation level (SpO₂)). The blood oxygen saturation level (SpO₂) is the ratio of the amount of oxygen actually bound to hemoglobin to the total amount of oxygen under the assumption that the oxygen is bound to all the hemoglobin in the blood.

As described above, the infrared light can be easily absorbed by hemoglobin. When the amount of hemoglobin increases, the amount of absorbed infrared light increase and the amounts of light received by the photodiodes PD decrease. That is, the total amount of hemoglobin is determined from the amount of the received reflected light L2 of the infrared light.

Meanwhile, the hemoglobin has a dark red color when not bound to oxygen, and has a bright red color when bound to oxygen. Therefore, the absorption coefficient of the hemoglobin for absorbing the red light differs between when the hemoglobin is bound to oxygen and when the hemoglobin is not bound to oxygen. As a result, the amount of the reflected light of the red light increases as the hemoglobin bound to oxygen increases in the blood. In contrast, the amount of the reflected light of the red light decreases as the hemoglobin not bound to oxygen increases in the blood. Thus, the amount of the hemoglobin bound to oxygen is relatively determined based on the amount of the received reflected light L2 of the red light.

Then, by comparing the determined total amount of the hemoglobin with the amount of the hemoglobin bound to oxygen, the ratio of the amount of oxygen actually bound to the hemoglobin (blood oxygen saturation level (SpO₂)) can be determined.

In the present disclosure, the wavelengths of the light emitted from the first and the second light sources 111 and 112 are not limited to the wavelengths exemplified above. The first light sources 111 only need to emit the infrared light having a wavelength of from 800 nm to less than 1000 nm. The second light sources 112 only need to emit the red light having a wavelength of from 600 nm to less than 800 nm. The following describes details of the detection device 1 of the first embodiment.

FIG. 2 is a plan view illustrating the detection device according to the first embodiment. As illustrated in FIG. 2, the detection device 1 includes the base member 21 (array substrate 2), a sensor 10, a gate line drive circuit 15, a signal line selection circuit 16, a detection circuit 48, a control circuit 53, and a power supply circuit 54.

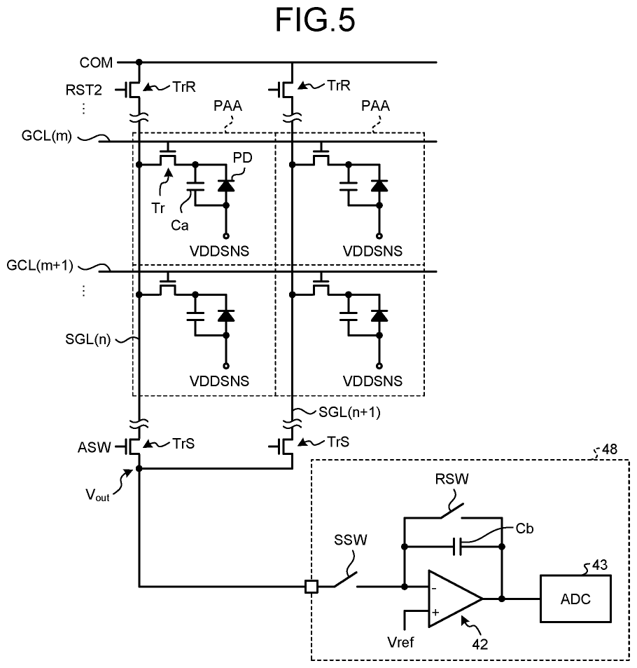

The base member 21 is electrically coupled to a control substrate 52 through a flexible printed circuit board 51. The flexible printed circuit board 51 is provided with the detection circuit 48. The control substrate 52 is provided with the control circuit 53 and the power supply circuit 54. The control circuit 53 is, for example, a field-programmable gate array (FPGA). The control circuit 53 supplies control signals to the sensor 10, the gate line drive circuit 15, and the signal line selection circuit 16 to control a detection operation of the sensor 10. The control circuit 53 supplies control signals to the backlight 101 (refer to FIG. 1) to control lighting and non-lighting of the light sources 110. The power supply circuit 54 supplies voltage signals including, for example, a sensor power supply signal (sensor power supply voltage) VDDSNS (refer to FIG. 5) to the sensor 10, the gate line drive circuit 15, and the signal line selection circuit 16. The power supply circuit 54 supplies a power supply voltage to the light sources 110.

The base member 21 has the detection area AA and the peripheral area GA. The detection area AA is an area provided with the photodiodes PD included in the sensor 10. The detection area AA of the present disclosure has a rectangular shape in plan view. The peripheral area GA is an area between the outer perimeter of the detection area AA and the ends of the base member 21. In other words, the peripheral area GA is an area where the photodiodes PD are not provided. The peripheral area GA of the present embodiment has a rectangular frame shape.

The gate line drive circuit 15 and the signal line selection circuit 16 are provided in the peripheral area GA. Specifically, the gate line drive circuit 15 is provided in an area extending along a second direction Dy in the peripheral area GA. The signal line selection circuit 16 is provided in an area extending along a first direction Dx in the peripheral area GA and is disposed between the sensor 10 and the detection circuit 48.

In the following description, the first direction Dx is one direction in a plane parallel to the base member 21. The second direction Dy is one direction in the plane parallel to the base member 21 and is a direction orthogonal to the first direction Dx. The second direction Dy may non-orthogonally intersect the first direction Dx. A third direction Dz is a direction orthogonal to the first direction Dx and the second direction Dy and is a direction normal to the first principal surface S1 of the base member 21. The term “plan view” refers, for example, to a positional relation when viewed from the object to be detected 200, that is, when viewed in a facing direction facing the first principal surface S1 of the base member 21. The term “when viewed in the facing direction” may be substituted for the term “in plan view”.

The sensor 10 includes the photodiodes PD arranged in the first and the second directions Dx and Dy. That is, the detection area AA is divided into a plurality of partial detection areas PAA corresponding to the respective photodiodes PD. Each of the photodiodes PD is a photoelectric conversion element and outputs an electrical signal corresponding to light received thereby. More specifically, the photodiode PD is an organic photodiode (OPD). The photodiodes PD are arranged in the first and the second directions Dx and Dy. That is, the photodiodes PD are arranged in a matrix having a row-column configuration. Therefore, the partial detection areas PAA are also divided into a matrix having a row-column configuration.

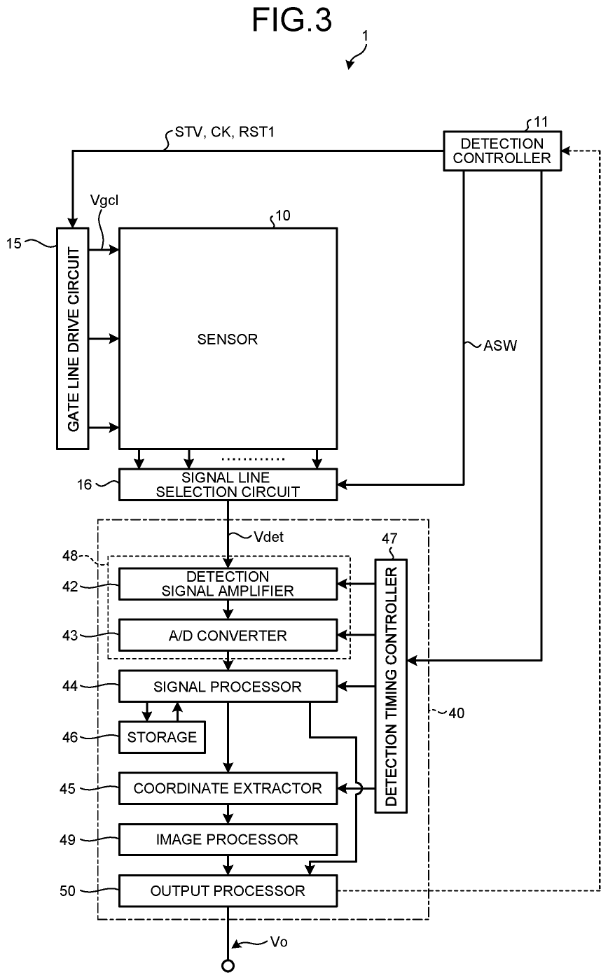

FIG. 3 is a block diagram illustrating a configuration example of the detection device according to the first embodiment. As illustrated in FIG. 3, the detection device 1 further includes a detection controller (detection control circuit) 11 and a detector (detection signal processing circuit) 40. The control circuit 53 includes one, some, or all functions of the detection controller 11. The control circuit 53 also includes one, some, or all functions of the detector 40 other than those of the detection circuit 48.

Each of the photodiodes PD included in the sensor 10 outputs an electrical signal corresponding to light emitted to the photodiode PD as a detection signal Vdet to the signal line selection circuit 16. The sensor 10 performs the detection according to a gate drive signal Vgcl supplied from the gate line drive circuit 15.

The detection controller 11 is a circuit that supplies respective control signals to the gate line drive circuit 15, the signal line selection circuit 16, and the detector 40 to control operations of these components. The detection controller 11 supplies various control signals including, for example, a start signal STV, a clock signal CK, and a reset signal RST1 to the gate line drive circuit 15. The detection controller 11 also supplies various control signals including, for example, a selection signal ASW to the signal line selection circuit 16. The detection controller 11 also supplies various control signals to the backlight 101 to control the lighting and non-lighting of the light sources 110.

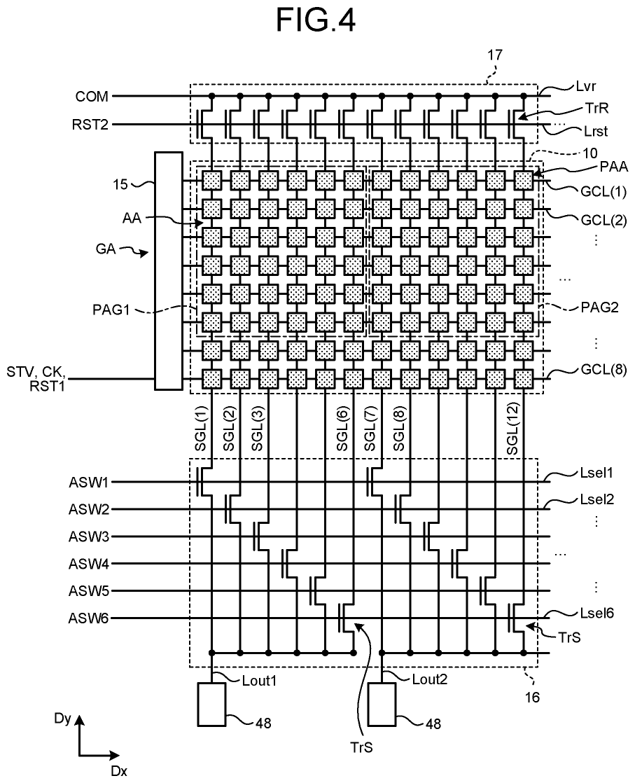

The gate line drive circuit 15 is a circuit that drives a plurality of gate lines GCL (refer to FIG. 4) based on the various control signals. The gate line drive circuit 15 sequentially or simultaneously selects the gate lines GCL. The gate line drive circuit 15 then supplies the gate drive signals Vgcl to the selected gate lines GCL. By this operation, the gate line drive circuit 15 selects the photodiodes PD coupled to the gate lines GCL.

The signal line selection circuit 16 is a switch circuit that sequentially or simultaneously selects a plurality of signal lines SGL (refer to FIG. 4). The signal line selection circuit 16 is, for example, a multiplexer. The signal line selection circuit 16 couples the selected signal lines SGL to the detection circuit 48 based on the selection signal ASW supplied from the detection controller 11. By this operation, the photodiodes PD output the detection signals Vdet to the detector 40.

The detector 40 includes the detection circuit 48, a signal processor (signal processing circuit) 44, a coordinate extractor (coordinate extraction circuit) 45, a storage (storage circuit) 46, a detection timing controller (detection timing control circuit) 47, an image processor (image processing circuit) 49, and an output processor (output processing circuit) 50. Based on a control signal supplied from the detection controller 11, the detection timing controller 47 controls the detection circuit 48, the signal processor 44, the coordinate extractor 45, and the image processor 49 so as to operate in synchronization with one another.

The detection circuit 48 is, for example, an analog front-end (AFE) circuit. The detection circuit 48 is a signal processing circuit having functions of at least a detection signal amplifier 42 and an analog-to-digital (A/D) converter 43. The detection signal amplifier 42 amplifies the detection signals Vdet. The A/D converter 43 converts analog signals output from the detection signal amplifier 42 into digital signals.

The signal processor 44 is a logic circuit that detects a predetermined physical quantity received by the sensor 10 based on an output signal of the detection circuit 48. The signal processor 44 detects the information on the blood based on the signals from the detection circuit 48 when a finger is in contact with or in proximity to a detection surface.

The signal processor 44 may also perform processing of acquiring the detection signals Vdet (blood information) simultaneously detected by the photodiodes PD, and averaging the detection signals Vdet. In this case, the detector 40 can perform stable detection by reducing measurement errors caused by noise or relative positional misalignment between the object to be detected, such as a finger, and the sensor 10.

The storage 46 temporarily stores therein signals calculated by the signal processor 44. The storage 46 may be, for example, a random-access memory (RAN) or a register circuit.

The coordinate extractor 45 is a logic circuit that obtains detected coordinates of blood vessels when the contact or the proximity of the object to be detected 200 is detected by the signal processor 44. The image processor 49 combines the detection signals Vdet output from the respective photodiodes PD of the sensor 10 to generate two-dimensional information indicating a blood vessel image (vascular pattern). The coordinate extractor 45 may output the detection signals Vdet as sensor output voltages Vo instead of calculating the detected coordinates. A case can be considered where the detector 40 does not include the coordinate extractor 45 and the image processor 49.

The output processor 50 serves as a processor that performs processing based on the outputs from the photodiodes PD. The output processor 50 may include, for example, the detected coordinates obtained by the coordinate extractor 45 and the two-dimensional information generated by the image processor 49 in the sensor output voltages Vo. The function of the output processor 50 may be integrated into another component (for example, the image processor 49).

The following describes a circuit configuration example of the detection device 1. FIG. 4 is a circuit diagram illustrating the detection device. As illustrated in FIG. 4, the sensor 10 has the partial detection areas PAA arranged in a matrix having a row-column configuration. Each of the partial detection areas PAA is provided with the photodiode PD.

The gate lines GCL extend in the first direction Dx, and are each coupled to the partial detection areas PAA arranged in the first direction Dx. A plurality of gate lines GCL(1), GCL(2), . . . , GCL(8) are arranged in the second direction Dy, and are each coupled to the gate line drive circuit 15. In the following description, the gate lines GCL(1), GCL(2), . . . , GCL(8) will each be simply referred to as the gate line GCL when they need not be distinguished from one another. For ease of understanding of the description, FIG. 4 illustrates eight gate lines GCL. However, this is merely an example, and M gate lines GCL may be arranged (where M is 8 or larger, and is, for example, 256).

The signal lines SGL extend in the second direction Dy and are each coupled to the photodiodes PD of the partial detection areas PAA arranged in the second direction Dy. A plurality of signal lines SGL(1), SGL(2), . . . , SGL(12) are arranged in the first direction Dx and are each coupled to the signal line selection circuit 16 and a reset circuit 17. In the following description, the signal lines SGL(1), SGL(2), . . . , SGL(12) will each be simply referred to as the signal line SGL when they need not be distinguished from one another.

For ease of understanding of the description, 12 of the signal lines SGL are illustrated. However, this is merely an example, and N signal lines SGL may be arranged (where N is 12 or larger, and is, for example, 252). The resolution of the sensor is, for example, 508 dots per inch (dpi), and the number of cells is 252×256. In FIG. 4, the sensor 10 is provided between the signal line selection circuit 16 and the reset circuit 17. The present disclosure is not limited to this configuration. The signal line selection circuit 16 and the reset circuit 17 may be coupled to ends of the signal lines SGL in the same direction.

The gate line drive circuit 15 receives the various control signals such as the start signal STV, the clock signal CK, and the reset signal RST1 from the control circuit 53 (refer to FIG. 2). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr (refer to FIG. 5) coupled to the gate line GCL, and corresponding ones of the partial detection areas PAA arranged in the first direction Dx are selected as detection targets.

The signal line selection circuit 16 includes a plurality of selection signal lines Lsel, a plurality of output signal lines Lout, and third switching elements TrS. The third switching elements TrS are provided correspondingly to the signal lines SGL. Six signal lines SGL(1), SGL(2), . . . , SGL(6) are coupled to a common output signal line Lout1. Six signal lines SGL(7), SGL(8), . . . , SGL(12) are coupled to a common output signal line Lout2. The output signal lines Lout1 and Lout2 are each coupled to the detection circuit 48.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the respective third switching elements TrS included in one of the signal line blocks. One of the selection signal lines Lsel is coupled to the gates of the third switching elements TrS in the signal line blocks.

The control circuit 53 (refer to FIG. 2) sequentially supplies the selection signal ASW to the selection signal lines Lsel. This operation causes the signal line selection circuit 16 to operate the third switching elements TrS to sequentially select the signal lines SGL in one of the signal line blocks in a time-division manner. The signal line selection circuit 16 selects one of the signal lines SGL in each of the signal line blocks. The above-described configuration can reduce the number of integrated circuits (ICs) including the detection circuit 48 or the number of terminals of the ICs in the detection device 1. The signal line selection circuit 16 may collectively couple more than one of the signal lines SGL to the detection circuit 48.

As illustrated in FIG. 4, the reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 53 supplies a reset signal RST2 to the reset signal line Lrst. This operation turns on the fourth switching elements TrR to electrically couple the signal lines SGL to the reference signal line Lvr. The power supply circuit 54 supplies a reference signal COM to the reference signal line Lvr. This operation supplies the reference signal COM to a capacitive element Ca (refer to FIG. 5) included in each of the partial detection areas PAA.

FIG. 5 is a circuit diagram illustrating the partial detection areas. FIG. 5 also illustrates a circuit configuration of the detection circuit 48. As illustrated in FIG. 5, each of the partial detection areas PAA includes the photodiode PD, the capacitive element Ca, and a corresponding one of the first switching elements Tr. The capacitive element Ca is capacitance (sensor capacitance) generated in the photodiode PD, and is equivalently coupled in parallel to the photodiode PD.

FIG. 5 illustrates two gate lines GCL(m) and GCL(m+1) arranged in the second direction Dy among the gate lines GCL. FIG. 5 also illustrates two signal lines SGL(n) and SGL(n+1) arranged in the first direction Dx among the signal lines SGL. The partial detection area PAA is an area surrounded by the gate lines GCL and the signal lines SGL.

The first switching elements Tr are provided correspondingly to the photodiodes PD. Each of the first switching elements Tr is formed of a thin-film transistor, and in this example, formed of an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the photodiode PD and the capacitive element Ca.

The anode of the photodiode PD is supplied with the sensor power supply signal VDDSNS from the power supply circuit 54. The signal line SGL and the capacitive element Ca are supplied with the reference signal COM that serves as an initial potential of the signal line SGL and the capacitive element Ca from the power supply circuit 54.

When the partial detection area PAA is irradiated with light, a current corresponding to the amount of the light flows through the photodiode PD. As a result, an electric charge is stored in each of the capacitive element Ca. After the first switching element Tr is turned on, a current corresponding to the electric charge stored in the capacitive element Ca flows through the signal line SGL. The signal line SGL is coupled to the detection circuit 48 through a corresponding one of the third switching elements TrS of the signal line selection circuit 16. Thus, the detection device 1 can detect a signal corresponding to the amount of the light received by the photodiode PD in each of the partial detection areas PAA or in each block.

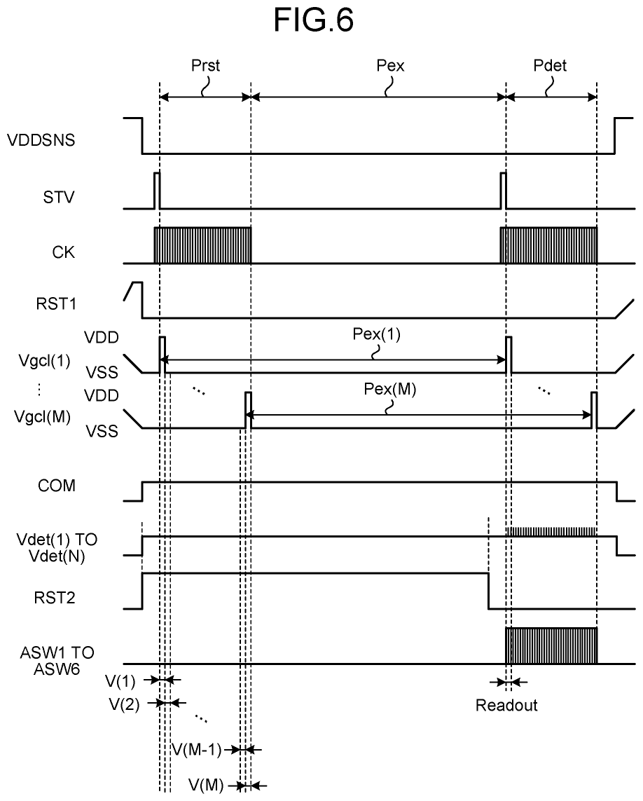

During a read period Pdet (refer to FIG. 6), a switch SSW is turned on to couple the detection circuit 48 to the signal lines SGL. The detection signal amplifier 42 of the detection circuit 48 converts a current supplied from the signal lines SGL into a voltage corresponding to the value of the current, and amplifies the result. A reference potential (Vref) having a fixed potential is supplied to a non-inverting input terminal (+) of the detection signal amplifier 42, and the signal lines SGL are coupled to an inverting input terminal (−) of the detection signal amplifier 42. In the embodiment, the same signal as the reference signal COM is supplied as the reference potential (Vref) voltage. The signal processor 44 (refer to FIG. 3) calculates the difference between the detection signal Vdet when light is emitted to the photodiode PD and the detection signal Vdet when light is not emitted to the photodiode PD as each of the sensor output voltages Vo. The detection signal amplifier 42 includes a capacitive element Cb and a reset switch RSW. During a reset period Prst (refer to FIG. 6), the reset switch RSW is turned on, and the electric charge of the capacitive element Cb is reset.

The following describes an operation example of the detection device 1. In the operation example of the detection device 1, an exemplary case will be described where the detection signals Vdet of all the partial detection areas PAA are output to the detection circuit 48.

FIG. 6 is a timing waveform diagram illustrating the operation example of the detection device. As illustrated in FIG. 6, the detection device 1 has a reset period Prst, an exposure period Pex, and the read period Pdet. The power supply circuit 54 supplies the sensor power supply signal VDDSNS to the anode of the photodiode PD over the reset period Prst, the exposure period Pex, and the read period Pdet. The sensor power supply signal VDDSNS is a signal that applies a reverse bias between the anode and the cathode of the photodiode PD. For example, the reference signal COM of substantially 0.75 V is applied to the cathode of the photodiode PD, and the sensor power supply signal VDDSNS of substantially −1.25 V is applied to the anode thereof. As a result, a reverse bias of substantially 2.0 V is applied between the anode and the cathode. The control circuit 53 sets the reset signal RST2 to “H”, and then, supplies the start signal STV and the clock signal CK to the gate line drive circuit 15 to start the reset period Prst. During the reset period Prst, the control circuit 53 supplies the reference signal COM to the reset circuit 17, and uses the reset signal RST2 to turn on the fourth switching elements TrR for supplying a reset voltage. This operation supplies the reference signal COM as the reset voltage to each of the signal lines SGL. The reference signal COM is set to, for example, 0.75 V.

During the reset period Prst, the gate line drive circuit 15 sequentially selects each of the gate lines GCL based on the start signal STV, the clock signal CK, and the reset signal RST1. The gate line drive circuit 15 sequentially supplies gate drive signals Vgcl {Vgcl(1), . . . , Vgcl(M)} to the gate lines GCL. Each of the gate drive signals Vgcl has a pulsed waveform having a power supply voltage VDD serving as a high-level voltage and a power supply voltage VSS serving as a low-level voltage. In FIG. 6, M gate lines GCL (where M is, for example, 256) are provided, and the gate drive signals Vgcl(1), . . . , Vgcl(M) are sequentially supplied to the respective gate lines GCL. Thus, the first switching elements Tr are sequentially brought into a conducting state and supplied with the reset voltage on a row-by-row basis. For example, a voltage of 0.75 V of the reference signal COM is supplied as the reset voltage.

Through this operation, during the reset period Prst, the capacitive elements Ca of all the partial detection areas PAA are sequentially electrically coupled to the signal lines SGL, and are supplied with the reference signal COM. As a result, the capacitance of the capacitive elements Ca is reset. The capacitance of the capacitive elements Ca of some of the partial detection areas PAA can be reset by partially selecting the gate lines and the signal lines SGL.

Examples of the method of controlling the exposure include a method of controlling the exposure during non-selection of the gate lines and a method of always controlling the exposure. In the method of controlling the exposure during non-selection of the gate lines, the gate drive signals Vgcl {Vgcl(1), . . . , Vgcl(M)} are sequentially supplied to all the gate lines GCL coupled to the photodiodes PD serving as the detection targets, and all the photodiodes PD serving as the detection targets are supplied with the reset voltage. Then, after all the gate lines GCL coupled to the photodiodes PD serving as the detection targets are set to a low voltage (the first switching elements Tr are turned off), the actual exposure starts and the actual exposure is performed during the exposure period Pex. After the actual exposure ends, the gate drive signals Vgcl {Vgcl(1), . . . , Vgcl(M)} are sequentially supplied to the gate lines GCL coupled to the photodiodes PD serving as the detection targets as described above, and reading is performed during the read period Pdet.

In the method of always controlling the exposure, the control for performing the exposure can also be performed during the reset period Prst and the read period Pdet (the exposure is always controlled). In this case, the exposure period Pex(1) starts after the gate drive signal Vgcl(1) is supplied to the gate line GCL during the reset period Prst. Exposure periods Pex{(1), . . . , (M)} are periods during which the capacitive elements Ca are charged from the photodiodes PD. The electric charges stored in the capacitive elements Ca during the reset period Prst flow as reverse directional currents (from cathodes to anodes) through the photodiodes PD due to light emission, and potential differences in the capacitive elements Ca decrease. The start timing and the end timing of the actual exposure periods Pex(1), . . . , Pex(M) are different among the partial detection areas PAA corresponding to the gate lines GCL. Each of the exposure periods Pex(1), . . . , Pex(M) starts when the gate drive signal Vgcl changes from the power supply voltage VDD serving as the high-level voltage to the power supply voltage VSS serving as the low-level voltage during the reset period Prst. Each of the exposure periods Pex(1), . . . , Pex(M) ends when the gate drive signal Vgcl changes from the power supply voltage VSS to the power supply voltage VDD during the read period Pdet. The lengths of the exposure periods Pex(1), . . . , Pex(M) are equal.

In the method of controlling the exposure during non-selection of the gate lines, a current flows correspondingly to the light emitted to the photodiode PD in each of the partial detection areas PAA during the exposure periods Pex{(1) . . . (M)}. As a result, an electric charge is stored in each of the capacitive elements Ca.

At a time before the read period Pdet starts, the control circuit 53 sets the reset signal RST2 to a low-level voltage. This operation stops the operation of the reset circuit 17. The reset signal may be set to a high-level voltage only during the reset period Prst. During the read period Pdet, the gate line drive circuit 15 sequentially supplies the gate drive signals Vgcl(1), . . . , Vgcl(M) to the gate lines GCL in the same manner as during the reset period Prst.

Specifically, the gate line drive circuit 15 supplies the gate drive signal Vgcl(1) at the high-level voltage (power supply voltage VDD) to the gate line GCL(1) during a period V(1). The control circuit 53 sequentially supplies selection signals ASW1, . . . , ASW6 to the signal line selection circuit 16 during a period in which the gate drive signal Vgcl(1) is at the high-level voltage (power supply voltage VDD). This operation sequentially or simultaneously couples the signal lines SGL of the partial detection areas PAA selected by the gate drive signal Vgcl(1) to the detection circuit 48. As a result, the detection signal Vdet for each of the partial detection areas PAA is supplied to the detection circuit 48.

In the same manner, the gate line drive circuit 15 supplies the gate drive signals Vgcl(2), . . . , Vgcl(M−1), Vgcl(M) at the high-level voltage to gate lines GCL(2), . . . , GCL(M−1), GCL(M) during periods V(2), . . . , V(M−1), V(M), respectively. That is, the gate line drive circuit 15 supplies the gate drive signal Vgcl to the gate line GCL during each of the periods V(1), V(2), . . . , V(M−1), V(M). The signal line selection circuit 16 sequentially selects each of the signal lines SGL based on the selection signal ASW in each period in which the gate drive signal Vgcl is set to the high-level voltage. The signal line selection circuit 16 sequentially couples each of the signal lines SGL to one detection circuit 48. Thus, the detection device 1 can output the detection signals Vdet of all the partial detection areas PAA to the detection circuit 48 during the read period Pdet.

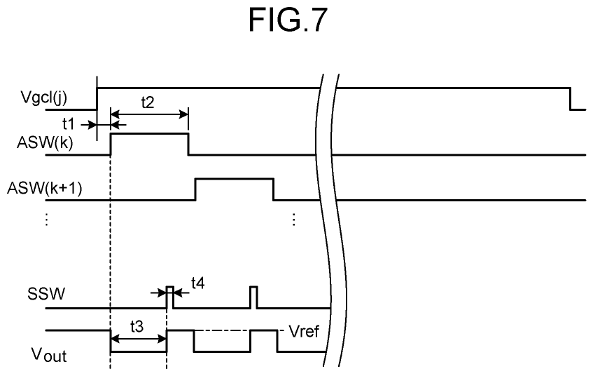

FIG. 7 is a timing waveform diagram illustrating an operation example during the read period in FIG. 6. With reference to FIG. 7, the following describes the operation example during a supply period Readout of one gate drive signal Vgcl(j) in FIG. 6. In FIG. 6, the reference numeral of the supply period Readout is assigned to the first gate drive signal Vgcl(1), and the same applies to the other gate drive signals Vgcl(2), . . . , Vgcl(M). The index j is any one of the natural numbers 1 to M.

As illustrated in FIGS. 7 and 5, an output voltage (Vₒᵤₜ) of each of the third switching elements TrS has been reset to the reference potential (Vref) voltage in advance. The reference potential (Vref) voltage serves as the reset voltage, and is set to 0.75 V, for example. Then, the gate drive signal Vgcl(j) is set to a high level, and the first switching elements Tr of a corresponding row are turned on. Thus, each of the signal lines SGL in each row is set to a voltage corresponding to the electric charge stored in the capacitor (capacitive element Ca) of the partial detection area PAA. After a period t1 elapses from a rising edge of the gate drive signal Vgcl(j), a period t2 starts in which the selection signal ASW(k) is set to a high level. After the selection signal ASW(k) is set to the high level and the third switching element TrS is turned on, the electric charge stored in the capacitor (capacitive element Ca) of the partial detection area PAA coupled to the detection circuit 48 through the third switching element TrS changes the output voltage (Vₒᵤₜ) of the third switching element TrS (refer to FIG. 5) to a voltage corresponding to the electric charge stored in the capacitor (capacitive element Ca) of the partial detection area PAA (period t3). In the example of FIG. 7, this voltage is reduced from the reset voltage as illustrated in the period t3. Then, after the switch SSW is turned on (in a period t4 during which an SSW signal is set to a high level), the electric charge stored in the capacitor (capacitive element Ca) of the partial detection area PAA moves to the capacitor (capacitive element Cb) of the detection signal amplifier 42 of the detection circuit 48, and the output voltage of the detection signal amplifier 42 is set to a voltage corresponding to the electric charge stored in the capacitive element Cb. At this time, the potential of the inverting input portion of the detection signal amplifier 42 is set to a virtual short-circuit potential of an operational amplifier, and therefore, becomes the reference potential (Vref). The A/D converter 43 reads the output voltage of the detection signal amplifier 42. In the example of FIG. 7, waveforms of the selection signals ASW(k), ASW(k+1), . . . corresponding to the signal lines SGL of the respective columns are set to a high level to sequentially turn on the third switching elements TrS, and the same operation is sequentially performed to sequentially read the electric charges stored in the capacitors (capacitive elements Ca) of the partial detection areas PAA coupled to the gate line GCL. ASW(k), ASW(k+1), . . . in FIG. 7 are, for example, any of ASW1 to ASW6 in FIG. 4.

Specifically, after the period t4 starts in which the switch SSW is on, the electric charge moves from the capacitor (capacitive element Ca) of the partial detection area PAA to the capacitor (capacitive element Cb) of the detection signal amplifier 42 of the detection circuit 48. At this time, the non-inverting input (+) of the detection signal amplifier 42 is set to the reference potential (Vref) voltage (for example, 0.75 V). As a result, the output voltage (Vₒᵤₜ) of the third switching element TrS is also set to the reference potential (Vref) voltage due to the virtual short-circuit between the input ends of the detection signal amplifier 42. The voltage of the capacitive element Cb is set to a voltage corresponding to the electric charge stored in the capacitor (capacitive element Ca) of the partial detection area PAA at a location where the third switching element TrS is turned on in response to the selection signal ASW(k). After the output voltage (V^{out}) of the third switching element TrS is set to the reference potential (Vref) due to the virtual short-circuit, the output voltage of the detection signal amplifier 42 reaches a voltage corresponding to the capacitance of the capacitive element Cb, and this output voltage is read by the A/D converter 43. The voltage of the capacitive element Cb is, for example, a voltage between two electrodes provided on a capacitor constituting the capacitive element Cb.

The period t1 is 20 μs, for example. The period t2 is 60 μs, for example. The period t3 is 44.7 μs, for example. The period t4 is 0.98 μs, for example.

Although FIGS. 6 and 7 illustrate the example in which the gate line drive circuit 15 individually selects the gate line GCL, the present disclosure is not limited to this example. The gate line drive circuit 15 may simultaneously select a predetermined number (two or more) of the gate lines GCL and sequentially supply the gate drive signals Vgcl to the gate lines GCL in units of the predetermined number of the gate lines GCL. The signal line selection circuit 16 may also simultaneously couple a predetermined number (two or more) of the signal lines SGL to one detection circuit 48. Moreover, the gate line drive circuit 15 may scan some of the gate lines GCL while skipping the others.

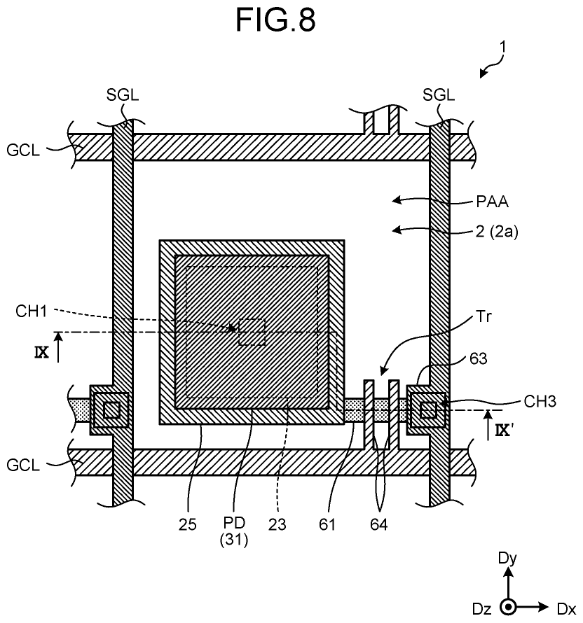

The following describes a configuration of the photodiode PD. FIG. 8 is a magnified schematic configuration diagram of the sensor. For ease of viewing, FIG. 8 illustrates an active layer 31 including an organic semiconductor material in a multilayered structure constituting the photodiode PD.

As illustrated in FIG. 8, the array substrate 2 includes various transistors such as the first switching elements Tr formed on the base member 21, the light shields 25, and various types of wiring such as the gate lines GCL and the signal lines SGL. In an area surrounded by the gate lines GCL and the signal lines SGL, the light-transmitting area 2a is an area that does not overlap the first switching element Tr and the light shield 25. Each of the light-blocking areas is an area overlapping the first switching element Tr and the light shield 25.

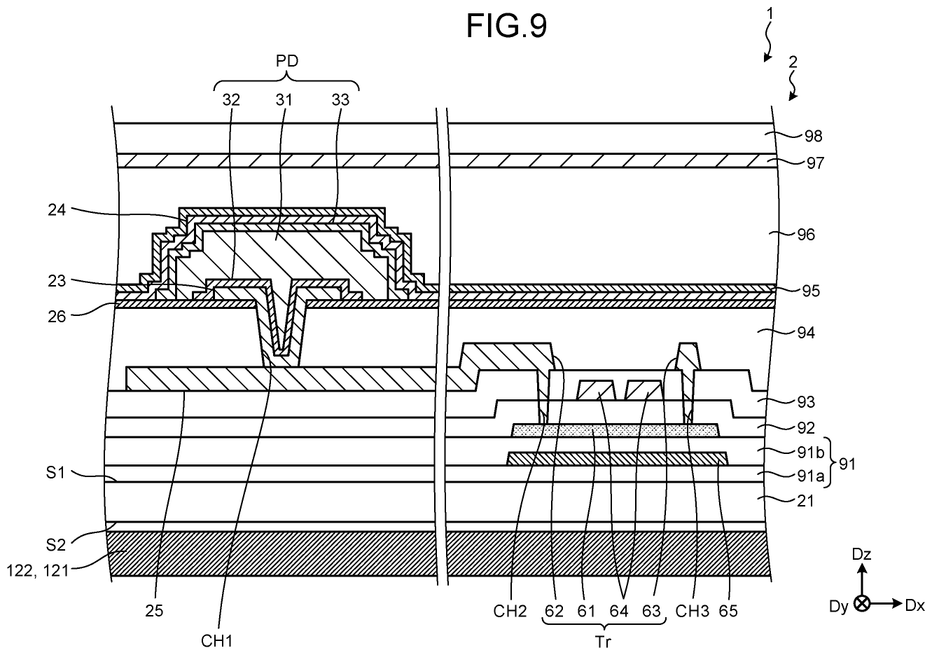

The first switching element Tr includes a semiconductor layer 61, a source electrode 62 (refer to FIG. 9), a drain electrode 63, and gate electrodes 64. The semiconductor layer 61 extends along the gate line GCL, and is provided so as to intersect the gate electrode 64 in plan view. The gate electrodes 64 are coupled to the gate line GCL, and extend in a direction orthogonal to the gate line GCL. One end side of the semiconductor layer 61 is coupled to the source electrode 62 (not illustrated in FIG. 8; refer to FIG. 9) through a second contact hole CH2 (refer to FIG. 9). The source electrode 62 is coupled to the light shield 25. The light shield 25 is electrically coupled to a first electrode 23 and the photodiode PD through a first contact hole CH1 formed in an organic insulating film 94 and a barrier film 26 (refer to FIG. 9). The other end side of the semiconductor layer 61 is coupled to the drain electrode 63 through a third contact hole CH3. The drain electrode 63 is coupled to the signal line SGL.

The configuration and the arrangement of the first switching element Tr illustrated in FIG. 8 are merely exemplary, and can be changed as appropriate. For example, the first switching element Tr has what is called a double-gate structure in which the two gate electrodes 64 are provided so as to intersect the semiconductor layer 61. However, one gate electrode 64 may be provided so as to intersect the semiconductor layer 61.

As illustrated in FIG. 8, the first electrode 23 and the photodiode PD are provided on the upper side of the light shield 25. The first electrode 23, the photodiode PD, and the light shield 25 are provided in an island shape in the area surrounded by the gate lines GCL and the signal lines SGL. In the present embodiment, the light-transmitting area 2a is formed around the first electrode 23, the photodiode PD, and the light shield 25 except in an area coupled to the first switching element Tr. The first electrodes 23 are provided correspondingly to the photodiodes PD in a matrix having a row-column configuration above the base member 21. The first electrodes 23 are each a cathode electrode of the photodiode PD, and may be called “detection electrode”.

In plan view, the area of the photodiode PD is smaller than the area of the light shield 25. The area of the first electrode 23 is smaller than the area of the active layer 31 that forms the photodiode PD. In addition, in plan view, the photodiode PD (active layer 31) is disposed inside the outer perimeter of the light shield 25. The first electrode 23 is disposed inside the outer perimeter of the photodiode PD.

With this configuration, in the detection device 1, the reflected light L2 that has been transmitted through the light-transmitting area 2a and reflected in the object to be detected 200 is emitted to the photodiodes PD. The detection device 1 can restrain the light L1 of the light from the backlight 101 that overlaps the light shields 25 from being emitted to the photodiodes PD.

The light shield 25, the first electrode 23, and the photodiode PD illustrated in FIG. 8 have each a quadrilateral shape. The shape of the light shield 25, the first electrode 23, and the photodiode PD is not limited to this shape, and may be another shape, such as a polygonal shape or a circular shape. The light shield 25, the first electrode 23, and the photodiode PD may have shapes different from one another. The areas, the shapes, and the arrangement pitch of the light shields 25, the first electrodes 23, and the photodiodes PD are merely exemplary and can be changed as appropriate depending on the characteristics and the detection accuracy required for the detection device 1.

FIG. 9 is a sectional view along IX-IX′ of FIG. 8. As illustrated in FIG. 9, the detection device 1 further includes a second electrode 24, an insulating film 95, a sealing film 96, and insulating films 97 and 98 that cover the photodiode PD. FIG. 9 does not illustrate a light guide 7 and the cover member 99 above the array substrate 2.

In the present specification, a direction from the base member 21 toward the photodiode PD along a direction orthogonal to a surface of the base member 21 is referred to as “upper side” or simply “above” or “on”. A direction from the photodiode PD toward the base member 21 is referred to as “lower side” or simply “below”.

The base member 21 is an insulating base material and is made using, for example, glass or a resin material. The base member 21 is not limited to having a flat plate shape and may have a curved surface. In this case, the base member 21 may be a film-like resin.

The base member 21 is provided with TFTs, such as the first switching elements Tr, and various types of wiring, such as the gate lines GCL and the signal lines SGL. The array substrate 2 obtained by forming the TFTs, the various types of wiring, and the photodiodes PD on the base member 21 is a drive circuit board for driving the sensor for each predetermined detection area and is also called a backplane or an active matrix substrate.

Undercoat films 91a and 91b are provided above the base member 21. A transistor light-blocking film 65 is provided above the base member 21 with the undercoat film 91a interposed therebetween. The transistor light-blocking film 65 is provided between the semiconductor layer 61 and the base member 21. The transistor light-blocking film 65 can restrain light from entering a channel area of the semiconductor layer 61 from the base member 21 side. Although FIG. 8 does not illustrate the transistor light-blocking film 65, the transistor light-blocking film 65 is as large as or larger than the semiconductor layer 61, when viewed in plan view.

The undercoat film 91b is provided above the base member 21 so as to cover the transistor light-blocking film 65. The undercoat films 91a and 91b are each formed of, for example, an inorganic insulating film such as a silicon nitride film or a silicon oxide film. An undercoat film 91 may be configured as a single-layer film in which the undercoat film 91a is not formed and only the undercoat film 91b is formed, or may be layered with a plurality of layers of three or more inorganic insulating films.

The first switching element Tr (transistor) is provided above the base member 21. The semiconductor layer 61 is provided on the undercoat film 91b. For example, polysilicon is used as the semiconductor layer 61. The semiconductor layer 61 is, however, not limited thereto and may be formed of, for example, a microcrystalline oxide semiconductor, an amorphous oxide semiconductor, or low-temperature polysilicon.

A gate insulating film 92 is provided on the undercoat film 91 so as to cover the semiconductor layer 61. The gate insulating film 92 is an inorganic insulating film, such as a silicon oxide film. The gate electrodes 64 are provided on the gate insulating film 92. In the example illustrated in FIG. 9, the first switching element Tr has a top-gate structure. However, the first switching element Tr is not limited thereto but may have a bottom-gate structure, or a dual-gate structure in which the gate electrodes 64 are provided on both the upper side and the lower side of the semiconductor layer 61.

An interlayer insulating film 93 is provided on the gate insulating film 92 so as to cover the gate electrodes 64. The interlayer insulating film 93 has, for example, a multilayered structure of a silicon nitride film and a silicon oxide film. The source electrode 62 and the drain electrode 63 are provided on the interlayer insulating film 93. The source electrode 62 is coupled to a source region of the semiconductor layer 61 through the second contact hole CH2 provided in the gate insulating film 92 and the interlayer insulating film 93. The drain electrode 63 is coupled to a drain region of the semiconductor layer 61 through the third contact hole CH3 provided in the gate insulating film 92 and the interlayer insulating film 93.

The light shield 25 is provided in the same layer as that of the source electrode 62 on the interlayer insulating film 93. In the present embodiment, the light shield 25 is formed continuously with, and of the same material as, the source electrode 62. In the present embodiment, the light shield 25 is a portion of the source electrode 62, and the light shield 25 and the source electrode 62 double as each other.

The organic insulating film 94 is provided on the interlayer insulating film 93 so as to cover the source electrode 62 and the drain electrode 63 of the first switching element Tr. The organic insulating film 94 is provided so as to further cover the light shield 25. The organic insulating film 94 is an organic planarizing film and has a better coverage property for steps formed by wiring and provides better surface flatness than inorganic insulating materials formed by, for example, chemical vapor deposition (CVD).

The barrier film 26 is provided on the organic insulating film 94. The barrier film 26 is an inorganic insulating film, for example. The first electrode 23, the photodiode PD, and the second electrode 24 are provided on the barrier film 26.

In more detail, the first electrodes 23 are arranged on the first principal surface S1 side of the base member 21 and are provided on the barrier film 26 so as to overlap the light shields 25. The first electrode 23 is the cathode electrode of the photodiode PD and is formed of, for example, a light-transmitting conductive material such as indium tin oxide (ITO). Alternatively, the detection device 1 is formed as a top-surface light receiving optical sensor including the backlight 101 as described above, and the first electrode 23 can be made using, for example, a metal material such as silver (Ag). Alternatively, the first electrode 23 may be made of a metal material such as aluminum (Al) or an alloy material containing at least one or more of these metal materials. As described above, the first electrodes 23 are arranged so as to be separated for each of the partial detection areas PAA (photodiodes PD).

The photodiode PD is provided so as to cover the first electrode 23. In more detail, the photodiode PD includes the active layer 31, an electron transport layer 32 (first carrier transport layer) provided between the active layer 31 and the first electrode 23, and a hole transport layer 33 (second carrier transport layer) provided between the active layer 31 and the second electrode 24. The photodiode PD is formed in an area overlapping the light shield 25 and has a configuration in which the first electrode 23, the electron transport layer 32, the active layer 31, the hole transport layer 33, and the second electrode 24 are stacked in this order in the direction orthogonal to the base member 21.

The active layer 31 changes in characteristics (for example, voltage-current characteristics and resistance value) according to light emitted thereto. An organic material is used as a material of the active layer 31. Specifically, the active layer 31 has a bulk heterostructure containing a mixture of a p-type organic semiconductor and an n-type fullerene derivative (PCBM) that is an n-type organic semiconductor. As the active layer 31, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 31 can be formed by a vapor deposition process (dry process) using the above-listed low-molecular-weight organic materials. In this case, the active layer 31 may be, for example, a multilayered film of CuPc and F₁₆CuPc, or a multilayered film of rubrene and C₆₀. The active layer 31 can also be formed by a coating process (wet process). In this case, the active layer 31 is made using a material obtained by combining the above-listed low-molecular-weight organic materials with high-molecular-weight organic materials. As the high-molecular-weight organic materials, for example, poly(3-hexylthiophene) (P3HT) and F8-alt-benzothiadiazole (F8BT) can be used. The active layer 31 can be a film made of a mixture of P3HT and PCBM, or a film made of a mixture of F8BT and PDI.

The electron transport layer 32 and the hole transport layer 33 are provided to facilitate electrons and holes generated in the active layer 31 to reach the first electrode 23 or the second electrode 24. The electron transport layer 32 is provided so as to cover the upper and side surfaces of the first electrode 23. Outer edges of the electron transport layer 32 are in contact with the barrier film 26 in positions outside the first electrode 23. Ethoxylated polyethylenimine (PEIE) is used as a material of the electron transport layer 32.

The active layer 31 is in direct contact with the top of the electron transport layer 32. The active layer 31 is provided so as to cover the upper and side surfaces of the electron transport layer 32. Outer edges of the active layer 31 are in contact with the barrier film 26 in positions outside the electron transport layer 32.

The hole transport layer 33 is in direct contact with the top of the active layer 31. The hole transport layer 33 is provided so as to cover the upper and side surfaces of the active layer 31. Outer edges of the hole transport layer 33 are in contact with the barrier film 26 in positions outside the active layer 31. The hole transport layer 33 is a metal oxide layer. For example, tungsten oxide (WO₃) or molybdenum oxide is used as the oxide metal layer.

The side surfaces of the first electrode 23, the electron transport layer 32, and the active layer 31 are covered by the hole transport layer 33 located in the uppermost layer of the photodiode PD. In more detail, the outer edges of the first electrode 23, the electron transport layer 32, the active layer 31, and the hole transport layer 33 are in contact with the same plane on the upper surface of the barrier film 26. The sides of the first electrode 23, the sides of the electron transport layer 32, the sides of the active layer 31, and the sides of the hole transport layer 33 are arranged in this order along the upper surface of the barrier film 26. The electron transport layer 32 and the hole transport layer 33 are arranged so as to be separated with the active layer 31 interposed therebetween.

The second electrode 24 is provided on the photodiode PD. In more detail, the second electrode 24 is provided above the barrier film 26 so as to cover the upper and side surfaces of the hole transport layer 33. The second electrode 24 is an anode electrode of the photodiode PD. Although FIGS. 8 and 9 illustrate one of the partial detection areas PAA (photodiodes PD), the second electrode 24 is continuously provided across the partial detection areas PAA (photodiodes PD). The second electrode 24 is formed of, for example, a light-transmitting conductive material such as ITO or indium zinc oxide (IZO).

With the above-described configuration, the electron transport layer 32, the active layer 31, and the hole transport layer 33 forming the photodiode PD are provided in an island shape individually in the area surrounded by the gate lines GCL and the signal lines SGL. Each layer of the photodiode PD is provided so as to cover the upper and side surfaces of a layer therebelow, and the second electrode 24 is provided so as to contact the hole transport layer 33 and so as not to contact the first electrode 23, the electron transport layer 32, and the active layer 31 that are located in layers lower than the hole transport layer 33. Therefore, in the present embodiment, short circuits between the anode and the cathode of the photodiode PD can be reduced even when the photodiode PD is formed individually for each of the partial detection areas PAA. More specifically, compared with a configuration in which the electron transport layer 32, the active layer 31, and the hole transport layer 33 included in the photodiode PD are stacked to have the same width, and the side surfaces of each of the layers are exposed, the side surfaces of the respective layers can be restrained from being electrically coupled to one another through the second electrode 24 covering the photodiode PD.

The insulating film 95 is provided so as to cover the second electrode 24. The insulating film 95 is an inorganic insulating film and is continuously provided across the partial detection areas PAA (photodiodes PD) so as to cover the entire second electrode 24.

The sealing film 96 is provided on the second electrode 24. An inorganic film, such as a silicon nitride film or an aluminum oxide film, or a resin film, such as an acrylic film, is used as the sealing film 96. The sealing film 96 is not limited to a single layer, but may be a multilayered film having two or more layers obtained by combining the inorganic film with the resin film mentioned above. The sealing film 96 well seals the photodiode PD, and thus can restrain water from entering the photodiode PD from the upper surface side thereof.

The insulating films 97 and 98 are provided so as to cover the sealing film 96. The insulating film 97 is an inorganic insulating film, for example. The insulating film 98 is an organic insulating film (resin layer), for example.

The materials and manufacturing methods of the electron transport layer 32, the active layer 31, and the hole transport layer 33 are merely exemplary, and other materials and manufacturing methods may be used. The insulating films 97 and 98 only need to be provided as required and can be omitted.

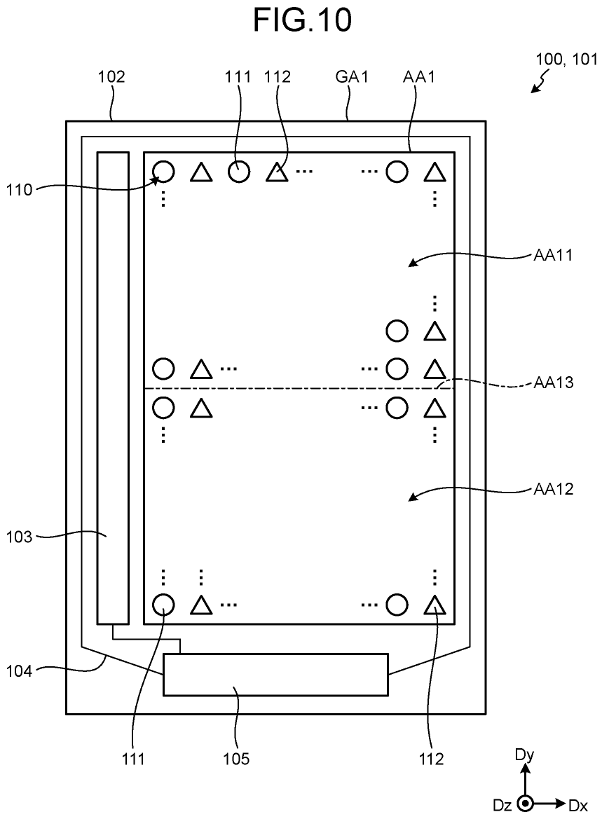

FIG. 10 is a plan view illustrating a configuration example of the light emission device according to the first embodiment. As illustrated in FIG. 10, the backlight 101 includes a substrate 102, a plurality of light sources 110, a drive circuit 103, cathode wiring 104, and a drive IC 105.

The substrate 102 is a drive circuit board for driving each of the light sources 110. The substrate 102 includes switch elements (such as transistors) and wiring such as signal lines (gate lines and signal lines), which are not illustrated, to drive the light sources. The substrate 102 has a light emission area AA1 corresponding to the detection area AA of the array substrate 2, and a peripheral area GA1 corresponding to the peripheral area GA of the array substrate 2.

The light emission area AA1 of the substrate 102 is provided with the light sources 110. The light sources 110 are arranged in the first and the second directions Dx and Dy to form a matrix having a row-column configuration. This configuration allows light to be emitted from a portion, instead of the entire area, of the light emission area AA1.

The light sources 110 include the first light sources 111 that emit the infrared light and the second light sources 112 that emit the red light. To easily distinguish the first light sources 111 from the second light sources 112, FIG. 10 illustrates the first light sources 111 in a circular shape and the second light sources 112 in a triangular shape. In the present disclosure, the shape of the first and the second light sources 111 and 112 in plan view is not limited.

Light-emitting diodes (LEDs) are used as the first and the second light sources 111 and 112. The first and the second light sources 111 and 112 are alternately arranged in the first direction Dx. In a column of the first light sources 111, the first light sources 111 are continuously arranged in the second direction Dy. In a column of the second light sources 112, the second light sources 112 are continuously arranged in the second direction Dy. In the present disclosure, the first and the second light sources 111 and 112 may be alternately arranged in the second direction Dy.

The size of the light source 110 in plan view is larger than that of the partial detection area PAA (refer to FIG. 8). In other words, when viewed in plan view, the light source 110 straddles one or both of the gate line GCL and the signal line SGL and overlaps more than one of the partial detection areas PAA. Therefore, when one of the light sources 110 is lit up, light passes through the light-transmitting area 2a of the partial detection areas PAA. In the present disclosure, the size of the light source 110 is not limited to this example. For example, the size of one light source 110 may fit into the size of the partial detection area PAA. Alternatively, a pair of the first and the second light sources 111 and 112 may have a size fitting into the partial detection area PAA.

The drive circuit 103 is disposed in the peripheral area GA1. The drive circuit 103 is a circuit that drives a plurality of gate lines based on various control signals from the drive IC 105. The drive IC 105 sequentially or simultaneously selects the gate lines and supplies gate drive signals to the selected gate lines.

The cathode wiring 104 is disposed in the peripheral area GA1. The cathode wiring 104 is disposed in the peripheral area GA1 of the substrate 21. The cathodes of the first and the second light sources are coupled to the cathode wiring through a common line, which is not illustrated, and supplied with a ground potential, for example.

The drive IC 105 is a circuit that controls the display of the backlight 101 by receiving various signals from the detection controller 11 of the array substrate 2. The drive IC 105 is mounted as a chip-on-glass (COG) in the peripheral area GA of the substrate 102. The drive IC 105 is not limited thereto, but may be mounted as a chip-on-film (COF) on a flexible printed circuit board or a rigid circuit board coupled to the peripheral area GA of the substrate 21.

The following describes an operation of the backlight 101 of the first embodiment during the detection of the blood information. The backlight 101 receives a control signal from the detection controller 11 of the array substrate 2 and lights up some of the light sources. That is, a lit area B1 where the backlight 101 (light emission device 100) is lit up during the detection is a portion of an area of the light sources 110, and the remaining portion forms an unlit area B2 where the light sources are not lit up. In other words, when viewed in the third direction Dz, a portion of the detection area AA overlapping the light sources 110 serves as the lit area B1. The portion of the detection area AA not overlapping the lit area B1 serves as the unlit area B2 where the backlight 101 (light emission device) is unlit.

The light emission area AA1 is divided into a first area AA11 disposed on one side and a second area AA12 disposed on the other side assuming an imaginary line AA13 extending in the first direction Dx through the center in the second direction Dy of the light emission area AA1 as a boundary. As a result, one of the first area AA11 and the second area AA12 serves as the lit area B1, while the other serves as the unlit area B2. Hereafter, a case where the first area AA11 is lit may be referred to as a first lighting time, and a case where the second area AA12 is lit may be referred to as a second lighting time.

If all the light sources 110 included in the first area AA11 are lit up at the first lighting time, it is not possible to determine whether the reflected light from the first light sources 111 or the second light sources 112 has been received by the photodiode PD. Therefore, at the first lighting time, the first light sources 111 are first lit up and the second light source 112 are unlit during that time. Then, the second light sources 112 are lit up and the first light sources 111 are unlit. After the photodiode PD has received the reflected light of the first light sources 111 but before the photodiode PD receives the reflected light of the second light sources 112, the electric charge of the capacitive element Cb (refer to FIG. 5) is once reset. The same operation is also performed at the second lighting time.

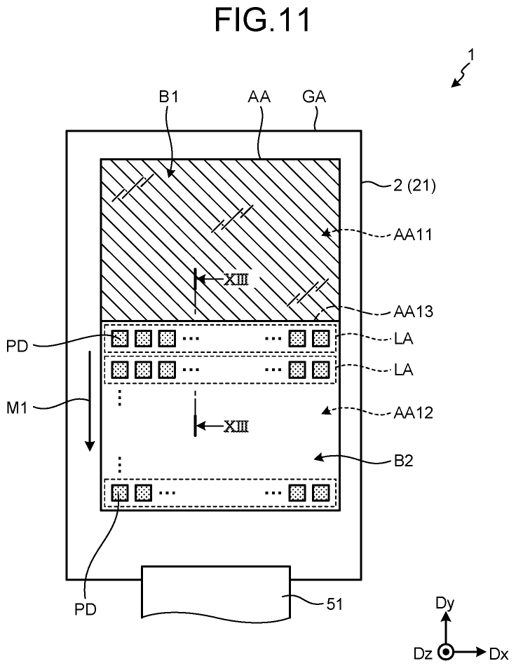

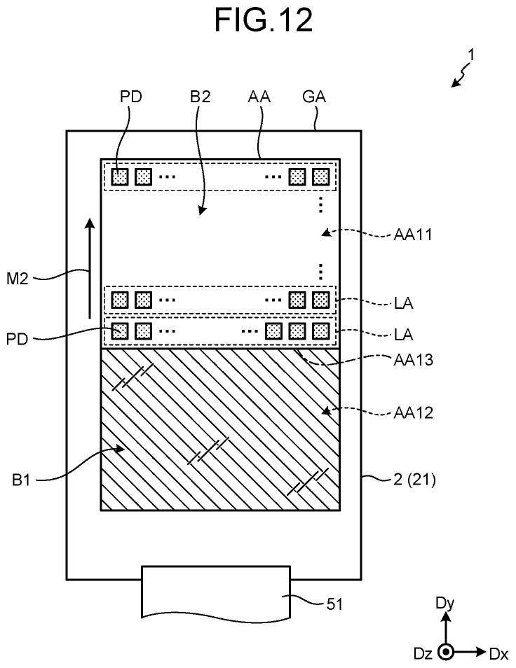

FIG. 11 is a plan view illustrating a reading area during the first lighting time in the first embodiment. FIG. 12 is a plan view illustrating the reading area at the second lighting time in the first embodiment.

The following describes a detection method of the detection device 1. The detection device 1 lights up the first area AA11 of the backlight 101 when detecting the blood information. The detection device 1 then selects a set of the photodiodes PD that are the photodiodes PD overlapping the second area AA12 (unlit area B2) in plan view and are arranged closest to the imaginary line AA13. The amounts of light received by the selected set of the photodiodes PD are then read. Hereafter, a group of the photodiodes PD each selected as a target of reading the amount of light received may be referred to as a reading area LA.

Then, another set of the photodiodes PD that are adjacent to the set of the photodiodes PD, the amounts of light received by which have been read, in a direction along the second direction Dy away from the imaginary line AA13 is selected and the amounts of light received thereby are read. Thus, the reading area LA is shifted in sequence in the second direction Dy (refer to an arrow M1 in FIG. 11). After the amounts of light received by all the photodiodes PD overlapping the second area AA12 (unlit area B2) have been read, the first area AA11 is unlit and the second area AA12 is lit up, as illustrated in FIG. 12.

At the second lighting time, the detection device 1 selects a set of the photodiodes PD that are the photodiodes PD overlapping the first area AA11 (unlit area B2) in plan view and are arranged closest to the imaginary line AA3. The amounts of light received by the selected set of the photodiodes PD are then read. Then, another set of the photodiodes PD that are adjacent to the set of the photodiodes PD, the amounts of light received by which have been read, in a direction along the second direction Dy away from the imaginary line AA13 is selected and the amounts of light received thereby are read. Thus, the reading area LA is shifted in sequence in the second direction Dy (refer to an arrow M2 in FIG. 12). After the amounts of light received by the all photodiodes PD overlapping the first area AA11 (unlit area B2) have been read, the detection of the blood information ends.

Thus, the detection device 1 of the first embodiment selects the photodiodes PD that overlap the unlit area B2 when viewed in the third direction Dz, and reads the amounts of light received by them. The following describes advantages of the detection device 1.

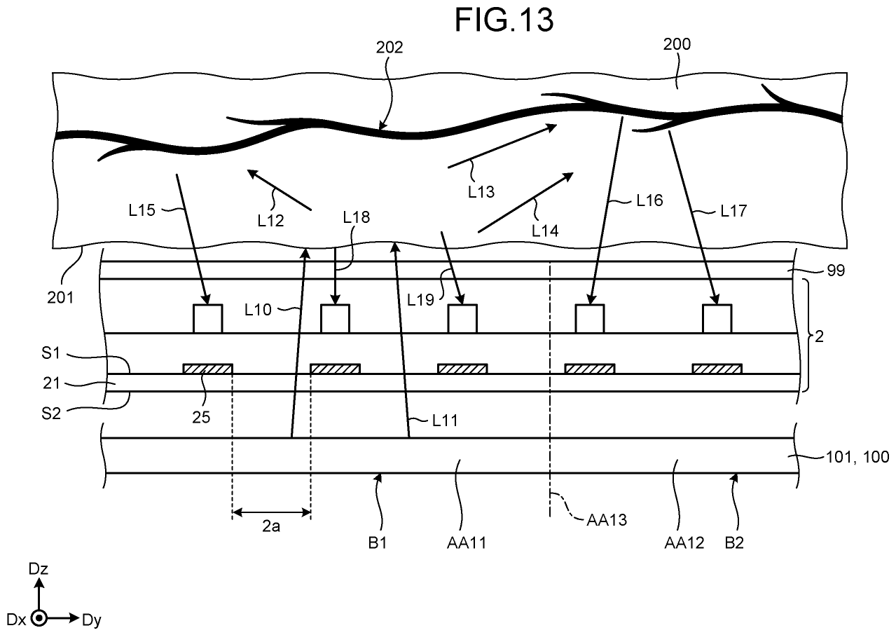

FIG. 13 is a sectional view at the first lighting time in the detection device of the first embodiment. As illustrated in FIG. 13, light is emitted from the first area AA11 of the backlight 101 at the first lighting time. The light passes through the light-transmitting area 2a of a portion of the array substrate 2 facing the first area AA11 (lit area B1) and is emitted to the skin 201 of the object to be detected 200 (refer to arrows L10 and L11 in FIG. 13). The light is not emitted to the second area AA12 (portion facing the unlit area B2) of the skin 201 of the object to be detected 200.

The light passes through the skin 201 and enters the object to be detected 200. The light that has entered the object to be detected 200 bends in the object to be detected 200 and diffuses toward directions of a plane parallel to the array substrate 2 (refer to arrows L12, L13, and L14). In other words, the light applied to the skin 201 linearly travels in the third direction Dz and bends in the object to be detected 200. That is, the traveling courses of the light are such that the light diffuses so as to includes, in addition to a component in the third direction Dz, components along directions of a plane parallel to the array substrate 2 such as a component in the first direction Dx and a component in the second direction Dy (refer to arrows L12, L13, and L14). As a result, the light passes through a portion in the object to be detected 200 that faces the second area AA12 (refer to the arrows L13 and L14).

The light is absorbed as it passes through the blood 202, and contains the blood information. The light is then reflected by the muscle tissues and the blood vessels (refer to arrows L15, L16, and L17) and emitted out of the object to be detected 200. The light emitted out of the object to be detected 200 is received by the photodiodes PD. The light has diffused in the object to be detected 200. The photodiodes PD that receive the light are not limited to those overlapping the first area AA11 (refer to the arrow L15), but also those overlapping the second area AA12 (refer to the arrows L16 and L17).

Part of the light emitted to the skin 201 of the object to be detected 200 (refer to the arrows L10 and L11 in FIG. 13) is reflected at the skin 201 (refer to an arrow L18). Alternatively, the light enters the object to be detected 200 from the skin 201, but is reflected at a shallow portion from the skin 201 (refer to an arrow L19). Such light reflected at the skin 201 and light reflected at the shallow portion from the skin 201 do not reach the blood 202 and do not contain the blood information. In addition, such light reflected at the skin 201 and light reflected at the shallow portion from the skin 201 have not fully diffused toward directions of the plane parallel to the array substrate 2 because of the shallow depth reached by the light that has entered the object to be detected 200. Therefore, the light reflected at the skin 201 and the light reflected at the shallow portion from the skin 201 are received by the photodiodes PD overlapping the first area AA11. In contrast, the photodiodes PD overlapping the unlit area B2 (second area AA12) in plan view is difficult to receive the light reflected at the skin 201 and the light reflected at the shallow portion from the skin 201 that serve as noise. Even if the light is received, the intensity of the reflected light is so weak that it is negligible in the detection of the blood information.

As described above, according to the detection device 1 of the first embodiment, when detecting the blood information, the photodiodes PD are difficult to receive the reflected light that serves as noise, and even if the reflected light is received, it can be ignored. Therefore, the blood information is highly reliable, and blood patterns and the blood oxygen saturation level (SpO₂) can be accurately detected.

The light shields 25 are arranged on the backlight 101 side of the photodiodes PD of the first embodiment. This configuration avoids direct reception of the light emitted from the backlight 101 by the photodiodes PD. Therefore, the accuracy of the blood patterns and the blood oxygen saturation level (SpO₂) detected is further improved.

### Second Embodiment

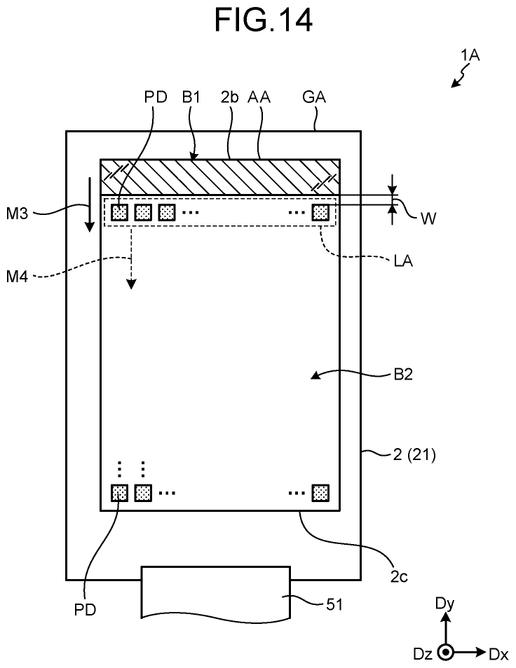

FIG. 14 is a plan view obtained by viewing a detection device according to a second embodiment of the present disclosure in plan view. The following describes a detection device 1A of the second embodiment. As illustrated in FIG. 14, the detection device 1A of the second embodiment differs from the detection device 1 of the first embodiment in terms of the method for lighting the backlight (refer to FIG. 10) during the detection.

In the backlight 101, among all the light sources 110, a set of the light sources 110 arranged in the first direction Dx is selected as the lit area B1 and lit up, and the lit area B1 is sequentially shifted in the second direction Dy. The starting position of the lit area B1 is at one end 2b of the detection area AA (light emission area AA1) (one end where the flexible printed circuit board 51 is not disposed) in the second direction Dy. The lit area B1 is then shifted toward the other end 2c of the detection area AA (light emission area AA1) (the other end where the flexible printed circuit board 51 is disposed) in the second direction Dy (refer to an arrow M3 in FIG. 14). By this method, light is emitted from the entire detection area AA (light emission area AA1).

Regarding the selection of the reading area LA, the detection device 1A selects, as the reading area LA, a set of the photodiodes PD adjacent to the lit area B1 in the second direction Dy and disposed on the other end 2c side of the lit area B1, and reads the amounts of light received thereby. Furthermore, in plan view, a predetermined gap W is provided between each of the photodiodes PD in the reading area LA and the lit area B1 in the second direction Dy. This gap W is 2 mm to 40 mm. If the gap W is smaller than 2 mm, the photodiodes PD may receive the light reflected at the skin 201 and the light reflected at the shallow portion from the skin 201. If, in contrast, the gap W exceeds 40 mm, the intensity of reflected light becomes weaker, which may reduce the accuracy of the blood information.

The reading area LA is shifted toward the other end 2c in the second direction Dy in synchronization with the shift of the lit area B1 (refer to the arrow M3 in FIG. 14) while maintaining the predetermined gap W. Then, the detection ends when the reading area LA has reached the other end 2c in the second direction Dy of the detection area AA.

Thus, the detection device 1A of the second embodiment selects the photodiodes PD that overlap the unlit area in plan view, and reads the amounts of light received by them. Therefore, in the same manner as in the first embodiment, the blood information is highly reliable, and the blood patterns and the blood oxygen saturation level (SpO₂) can be accurately detected. The gap W between the reading area LA and the lit area B1 is an adequate gap. Therefore, the accuracy of the blood patterns and the blood oxygen saturation level (SpO₂) is further improved.

According to the detection device 1A of the second embodiment, a set of the photodiodes PD arranged at the one end 2b in the second direction Dy of the detection area AA among the photodiodes PD overlaps the lit area B1 that is lit up first. Therefore, the amounts of light received by the set of the photodiodes PD arranged at the one end 2b in the second direction Dy of the detection area AA are not read. Therefore, in the present disclosure, the set of the photodiodes PD arranged at the one end 2b in the second direction Dy of the detection area AA and the set of the light sources 110 arranged at the other end 2c in the second direction Dy of the detection area AA may be omitted from the detection device.

### Third Embodiment

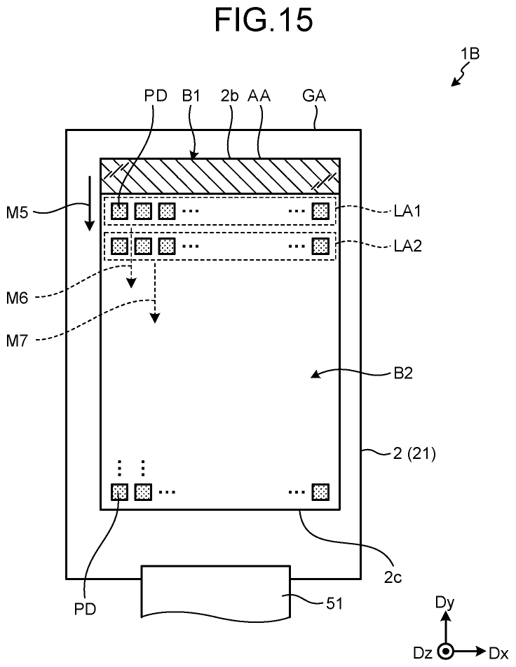

FIG. 15 is a plan view obtained by viewing a detection device according to a third embodiment of the present disclosure in plan view. A detection device 1B of the third embodiment differs from the detection device 1A of the second embodiment in terms of the selection of the reading area LA during the detection. The method for lighting by the detection device 1B of the third embodiment is such that the lit area B1 is shifted in sequence from the one end 2b toward the other end 2c of the detection area AA, in the same manner as in the second embodiment.

As illustrated in FIG. 15, the detection device 1B selects a reading area LA1 for the first light sources and a reading area LA2 for the second light sources. That is, the detection device 1B of the third embodiment selects two reading areas LA for one lit area B1.

The reading area LA1 for the first light sources is an area for reading the amount of reflected infrared light received. A set of the photodiodes PD adjacent to the lit area B1 and disposed on the other end 2c side of the lit area B1 is selected as the reading area LA1 for the first light sources. The reading area LA2 for the second light sources is an area for reading the amount of reflected red light received. A set of the photodiodes PD adjacent to the reading area LA1 and disposed on the other end 2c side of the reading area LA1 for the first light sources is selected as the reading area LA2 for the second light sources.

The reading area LA1 for the first light sources and the reading area LA2 for the second light sources are shifted toward the other end 2c in the second direction Dy (refer to arrows M6 and M7 in FIG. 15) in synchronization with the shift of the lit area B1 (refer to an arrow M5 in FIG. 15). This operation is repeated, and the detection is completed when the reading area LA2 for the second light sources has reached the other end 2c of the detection area AA.

Regarding the timing of reading the reading area LA1 for the first light sources and the reading area LA2 for the second light sources, the amounts of light received in the reading area LA1 for the first light sources are read after the first light sources 111 included in the lit area B1 are lit up. At this time, the amounts of light received in the reading area LA2 for the second light sources are not read. When the first light sources 111 have become unlit, the amounts of light received by all the photodiodes PD (the electric charge of the capacitive element Cb (refer to FIG. 5)) are reset. The second light sources 112 included in the lighting area B1 are then lit up, and the amounts of light received in the reading area LA2 for the second light sources are read. Thus, the reading area LA1 for the first light sources receives only the reflected infrared light, and the reading area LA2 for the second light sources receives only reflected red light.

As described above, the detection device 1B of the third embodiment selects the photodiodes PD that overlap the unlit area in plan view, and reads the amounts of light received by them. Therefore, in the same manner as in the first embodiment, the blood information is highly reliable, and the blood patterns and the blood oxygen saturation level (SpO₂) can be accurately detected.

### Fourth Embodiment

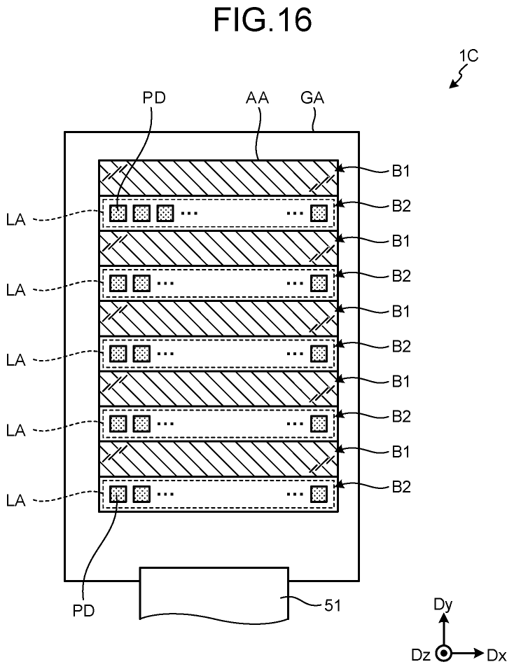

FIG. 16 is a plan view obtained by viewing a detection device according to a fourth embodiment of the present disclosure in plan view. A detection device 1C of the fourth embodiment will be described. The detection device 1C of the fourth embodiment differs from the detection device 1 of the first embodiment in terms of the method for lighting the backlight during the detection. The detection device 1C of the fourth embodiment also differs from the detection device 1 of the first embodiment in terms of the selection of the reading area during the detection.

As illustrated in FIG. 16, in the fourth embodiment, a set of the light sources 110 arranged in the first direction Dx is lit up and another set of the light sources 110 adjacent in the second direction Dy to the set of the light sources 110 is unlit. Thus, the lighting is performed such that the lit area B1 and the unlit area B2 are alternately provided in the second direction Dy. The detection device 1B selects the photodiodes PD overlapping the multiple unlit areas B2 as the reading areas LA and reads the amounts of light received thereby sequentially in the second direction Dy.

Then, although not illustrated, the light sources 110 currently included in the lit area B1 are unlit and the light sources 110 included in the unlit area B2 are lit up. That is, the lit area B1 and the unlit area B2 are interchanged. The detection device 1 then selects the photodiodes PD that newly overlap the unlit area B2 as the reading area and reads the amounts of light received thereby. As described above, also according to the fourth embodiment, the blood information is highly reliable, and the blood patterns and the blood oxygen saturation level (SpO₂) can be accurately detected.

### Fifth Embodiment

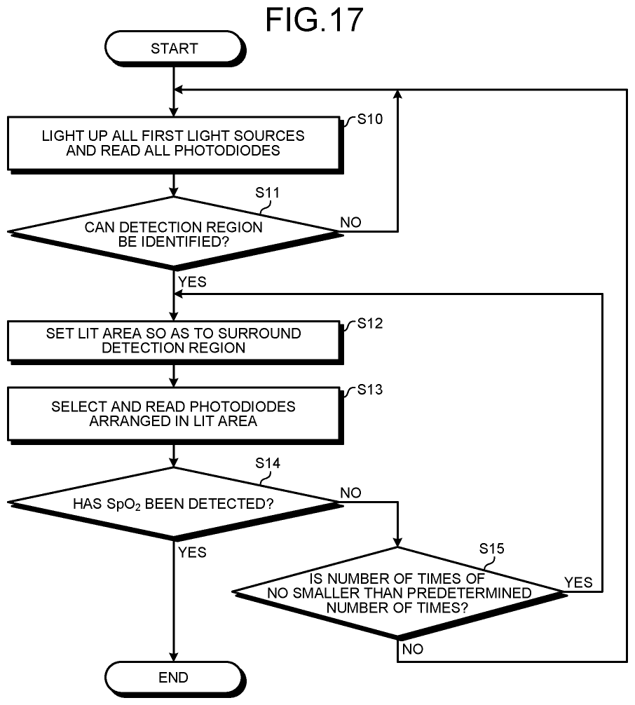

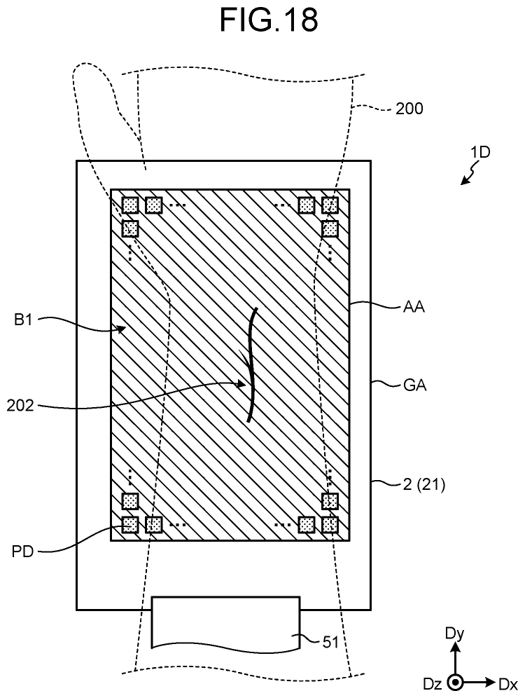

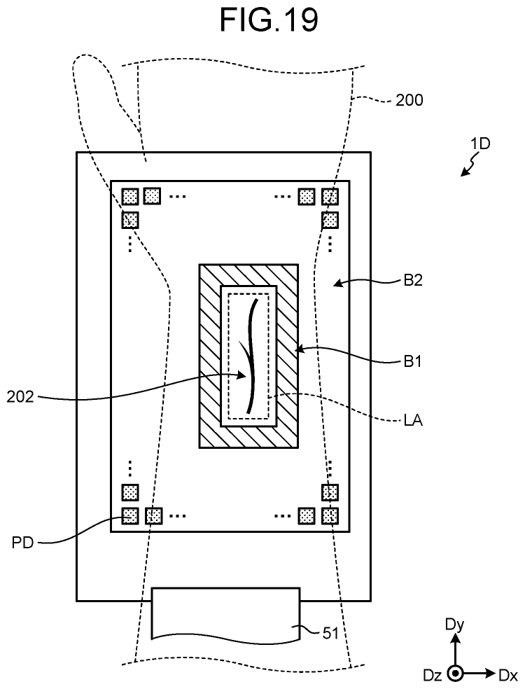

FIG. 17 is a flowchart for detecting the blood oxygen saturation level in a detection device according to a fifth embodiment of the present disclosure. FIG. 18 is a plan view obtained by viewing the detection device at a first step in plan view in the fifth embodiment. FIG. 19 is a plan view obtained by viewing the detection device at a third step in plan view in the fifth embodiment. A detection device 1D of the fifth embodiment will be described below. The detection device 1D of the fifth embodiment differs from the detection devices of the other embodiments in that a portion of the detection area AA is specified as a detection region and the blood oxygen saturation level (SpO₂) is detected from that detection region.

As illustrated in FIG. 17, the detection device 1D lights up all the first light sources 111 as a first step S10. As the first step S10, the detection device 1D selects all the photodiodes PDs included in the detection area AA and reads the amounts of light received thereby. Specifically, as illustrated in FIG. 18, the infrared light is emitted from the entire detection area AA to the object to be detected 200. The reflected light reflected from the object to be detected 200 is received by the photodiodes PD. As a result, the blood information on the entire object to be detected 200 is obtained.

As a second step S11, the detection device 1D identifies the vascular pattern (blood 202) of the object to be detected 200 that serves as the detection region, from the amounts of light received by all the photodiodes PD. Specifically, the amounts of light received by all the photodiodes PD are read. Thus, the vascular pattern (blood 202) of the object to be detected 200 is detected. The detection device 1D then specifies the detected vascular pattern (blood 202) and the vicinity thereof as the detection region. In the present disclosure, a part of the detected vascular pattern (blood 202) may be specified as the detection region.

At the first step S10, the entire area of the detection area AA is set as the lit area B1, and the light reflected at the skin and the shallow portion from the skin is received by the photodiodes PD. Therefore, the blood 202 may not be identified. Thus, if the detection region cannot be identified (“No” at the second step S11), the process returns to the first step S10.

If the detection region has been identified (“Yes” at the second step S11), the process goes to a third step S12. At the third step S12, the detection device 1D sets the lit area B1 so as to surround the detection region. In the present embodiment, as illustrated in FIG. 19, the lit area B1 is lit in a quadrilateral frame shape. The light sources 110 are lit so that the detection region (a portion of blood vessels) is surrounded by the frame-shaped lit area B1.

Although the lit area B1 has a quadrilateral frame shape in the present embodiment, the present disclosure is not limited thereto. The shape may be circular or triangular, or may be a shape along a particular object. At the third step S12, the light sources 110 to be lit up are both the first light sources 111 and the second light sources 112, the second light sources 112 being lit up after the first light sources 111 are lit up.

At a fourth step S13, a region surrounded by the lit area B1 in plan view is selected as the reading area LA. That is, more than one of the photodiodes PD overlapping the detection region are selected from among the photodiodes PD, and the amounts of light received thereby are read. In order not to receive the light reflected at the skin and the shallow portion from the skin, the photodiodes PD located at a predetermined gap (2 mm to 40 mm) from the lit area B1 are preferably selected.

Then, at a fifth step S14, the blood oxygen saturation level (SpO₂) is detected from the received amounts of the reflected light of the first light sources 111 and the second light sources 112, and the process ends (“end”) after the blood oxygen saturation level (SpO₂) is detected.

If, in contrast, the blood oxygen saturation level (SpO₂) cannot be detected, the process goes to a sixth step S15. At the sixth step S15, a determination is made as to whether the number of times the blood oxygen saturation level (SpO₂) has failed to be detected (the number of times “No” has been determined at S14) is smaller than a predetermined number of times. If the number of times the blood oxygen saturation level has failed to be detected is larger than the predetermined number of times, the detected area is likely to be not optimal. Therefore, if the number of times the blood oxygen saturation level (SpO₂) has failed to be detected is the predetermined number of times or larger (if “No” at the sixth step S15), the process returns to the first step S10 to restart from the identification of the detection region.

If, in contrast, the number of times the blood oxygen saturation level (SpO₂) has failed to be detected is smaller than the predetermined number of times (if “Yes” at the sixth step S15), the process returns to the third step S12 to detect the blood oxygen saturation level (SpO₂) again.

As described above, according to the detection device of the fifth embodiment, since the blood oxygen saturation level is detected after identifying the detection region (blood pattern), the blood oxygen saturation level can be identified with higher accuracy.

Although all the first light sources are lit up at the first step in the fifth embodiment, the first light sources may be lit up and the photodiodes overlapping the unlit area may be selected to read the amounts of light received thereby, as illustrated in the first to the fourth embodiments. This method allows the highly accurate blood pattern to be obtained.

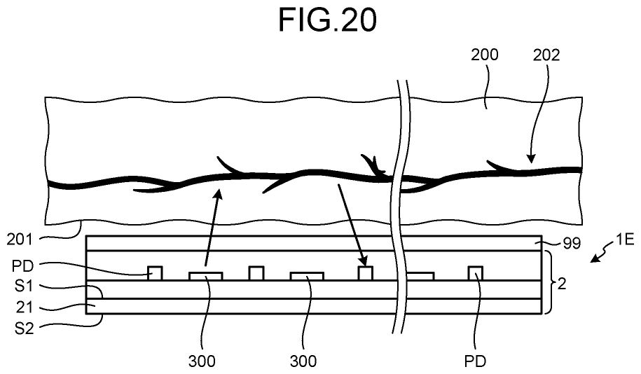

FIG. 20 is a sectional view of a detection device according to a modification. While the embodiments have been described above, the backlight 101 may be fixed to be in contact with the second principal surface S2 of the base member 21. The light emission device 100 may be a plurality of micro LEDs 300, as illustrated in FIG. 20. The micro LEDs 300 are arranged between the photodiodes PD. In the detection device 1E according to this modification, light is not emitted from the back side of the photodiodes PD (base member 21). Therefore, it is not necessary to provide the light shields 25.

In each of the embodiments, both the first and the second light sources are provided as the light sources 110. In the present disclosure, however, only the first light sources need to be provided. This is because the blood pattern can be detected if the detection device is provided with the first light sources. However, if the blood oxygen saturation level is to be detected as described in the fifth embodiment, both the first and the second light sources need to be provided.

What is claimed is:

1. A detection device comprising:
a base member having a first principal surface;
a detection area provided to the first principal surface;
a sensor including a plurality of photodiodes that are arranged in the detection area and arranged in a first direction parallel to the first principal surface and a second direction parallel to the first principal surface and intersecting the first direction;
a plurality of gate lines that extend in the first direction and are coupled to the photodiodes;
a plurality of signal lines that extend in the second direction and are coupled to the photodiodes; and
a light emission device configured to emit light through gaps between the photodiodes in a direction in which the first principal surface faces, wherein
the light emission device includes a plurality of light sources,
a lit area in which the light emission device is lit up is a portion of the detection area overlapping the light sources when viewed in a third direction intersecting the first direction and the second direction,
a portion of the detection area not overlapping the lit area is an unlit area in which the light emission device is not lit up, and
photodiodes that overlap the unlit area when viewed in the third direction are selected from among the photodiodes, and amounts of light received by the selected photodiodes are read.

2. The detection device according to claim 1, wherein the photodiodes from which the amounts of light received are read are separated by 2 mm to 40 mm from the lit area when viewed in the third direction.

3. The detection device according to claim 1, wherein
the light emission device has a first area disposed on one side and a second area disposed on the other side that are divided with a center in the second direction of the light emission device as a boundary therebetween, and
the first area and the second area are alternately lit up.

4. The detection device according to claim 1, wherein
the light sources are arranged in the first direction and the second direction,
from among the light sources, a set of light sources arranged in the first direction is selected as the lit area and lit up, and the lit area is sequentially shifted in the second direction and lit up, and
from among the photodiodes, a set of photodiodes that are adjacent in the second direction to the lit area and arranged in the first direction is selected as a reading area, the amounts of light received by the set of the photodiodes of the reading area are read, and the reading area is sequentially shifted in the second direction with the shift of the lit area.

5. The detection device according to claim 4, wherein
the lit area is shifted from one end toward the other end of the detection area in the second direction, and
the reading area is sequentially shifted from an area of a set of the photodiodes arranged on the other end side of the lit area in the second direction to the other end of the detection area in the second direction.

6. The detection device according to claim 5, wherein the detection device does not read the amounts of light received by a set of photodiodes arranged at the one end of the detection area in the second direction.

7. The detection device according to claim 1, wherein the light sources include first light sources that emit infrared light and second light sources that emit red light.

8. The detection device according to claim 7, wherein the first light sources and the second light sources are alternately arranged in the first direction or the second direction.

9. The detection device according to claim 1, wherein the detection device is configured to:
specify a portion of the detection area as a detection region; and
select the photodiodes that overlap the detection region when viewed in the third direction from among the photodiodes, and
read the amounts of light received by the selected photodiodes.

10. The detection device according to claim 9, wherein the lit area has a frame shape surrounding the detection region when viewed in the third direction.

11. The detection device according to claim 10, wherein
the light sources include first light sources that emit first light and second light sources that emit second light,
the first light is emitted from an entire area of the detection area, and all the photodiodes receive reflected light of the first light,
a vascular pattern is detected by reading the amounts of light received by all the photodiodes,
a region of the detection area in which the vascular pattern is detected is specified as the detection region,
the first light sources and the second light sources included in the frame-shaped lit area are lit up, and
a blood oxygen saturation level is detected by reading the amounts of light received by the photodiodes overlapping the detection region.

12. The detection device according to claim 11, wherein
the first light is infrared light, and
the second light is red light.

13. The detection device according to claim 1, wherein
the base member has a second principal surface located opposite the first principal surface, and
the light emission device is a backlight that faces the second principal surface and emits light toward the second principal surface.

14. The detection device according to claim 13, further comprising a plurality of light shields interposed between the first principal surface and the photodiodes.

15. The detection device according to claim 1, wherein the light sources are arranged between the photodiodes.
